(19) United States
(12) Patent Application Publication
(10) Pub. No.: US 2011/0136899 A1
(43) Pub. Date: Jun. 9, 2011

(54) COMBINATION OF DRONEDARONE WITH AT LEAST ONE DIURETIC, AND THERAPEUTIC USE THEREOF

(75) Inventors: Davide RADZIK, Paris (FR); Martin VAN EICKELS, Berlin (DE)

(73) Assignee: SANOFI-AVENTIS, Paris (FR)

(21) Appl. No.: 12/903,374

(22) Filed: Oct. 13, 2010

Related U.S. Application Data
(63) Continuation of application No. PCT/FR2009/000450, filed on Apr. 16, 2009.
(60) Provisional application No. 61/045,999, filed on Apr. 18, 2008.

(30) Foreign Application Priority Data
Apr. 17, 2008 (FR) 0802128

(51) Int. Cl. A61K 31/343; A61P 9/00; A61P 7/10; A61P 7/00; A61P 9/12

(52) U.S. Cl. 514/469

(57) ABSTRACT

The disclosure relates to a combination of dronedarone or a pharmaceutically salt thereof with at least one diuretic, and to the therapeutic use thereof.

This application is a continuation of International Application No. PCT/FR2009/000450, filed Apr. 16, 2009, which is incorporated herein by reference in its entirety; which claims the benefit of U.S. Provisional Application No. 61/045,999, filed Apr. 18, 2008 and claims the benefit of priority of French Patent Application No. 0802128, filed Apr. 17, 2008.

The present invention relates to a combination of dronedarone, or a pharmaceutically acceptable salt thereof, with at least one diuretic, and to the therapeutic application thereof.

2-n-Butyl-3-[4-(3-di-n-butylaminopropoxy)benzoyl]-5-methylsulphonamidobenzofuran, or dronedarone, and pharmaceutically acceptable salts thereof are described in European Patent EP 0 471 609 B1.

Dronedarone blocks potassium, sodium and calcium channels and also has anti-adrenergic properties.

Dronedarone is an anti-arrhythmic that is effective in maintaining sinus rhythm in patients presenting atrial fibrillation or atrial flutter.

The applicant has, surprisingly, found that dronedarone significantly reduces cardiovascular hospitalizations and mortality in patients having a history of atrial fibrillation or of atrial flutter, by virtue of its ability to modulate the blood potassium level.

In fact, the use of benzofuran derivatives to reduce post-infarction mortality in patients having a reduced left ventricular function after myocardial infarction, without any rhythm disorder requiring an anti-arrhythmic treatment, is known from Patent Applications WO 98/40067 and WO 97/34597.

However, these applications neither disclose nor suggest the use of dronedarone to reduce cardiovascular hospitalizations and/or mortality in patients having a history of atrial fibrillation or atrial flutter, in particular by virtue of its ability to modulate the potassium level in the blood.

Potassium is the principal intracellular ion and plays an essential role in physiology.

Specifically, this ion is the principal osmotically active intracellular ion and plays an important role in the regulation of intracellular volume.

A constant and stable potassium concentration is essential for the function of enzyme systems and also for good growth and cell division.

Potassium contributes to establishing the resting potential of the cell membrane and, consequently, changes in potassium concentration, in particular in the extracellular compartment, have effects on cell excitability in the nervous, muscle and cardiac system.

A decrease in potassium concentration is known to increase cardiac hyperexcitability at the ventricular level, which can result in serious, potentially deadly, rhythm disorders.

The deleterious role of a decrease in potassium concentration has been documented in disparate clinical situations.

For example, in patients suffering from heart failure, the decrease in potassium concentration can lead to deadly rhythm disorders; diuretics having a “potassium sparing” effect have demonstrated a beneficial effect in this population.

The rapid decrease in potassium concentrations occurring following the abrupt arrest of intense physical exercise could also be responsible for certain sudden deaths.

A possible contribution of the decrease in potassium concentrations has been mentioned in the sudden death of patients treated with antipsychotics and also in acute alcohol withdrawal syndromes.

Eating habits with a reduced potassium intake may lead to sudden death in predisposed individuals, even without any structural cardiac pathology.

The risk of fatal cardiac hyperexcitability is particularly great in patients who receive an anti-arrhythmic treatment which prolongs the duration of cell repolarization, such as sotalol (Sotalex®). These agents may in fact induce a torsade de pointe, which is a severe and potentially deadly ventricular tachycardia. Torsades de pointes are facilitated by the decrease in potassium concentration.

Finally, it has been shown that the decrease in potassium concentration induces atrial fibrillations (Manoach M., J. Mol. Cell. Cardiol., 1998, 30(6): A4[8]).

Another clinical situation where the risk of potentially fatal cardiac rhythm disorders is high is represented by patients treated with diuretics, these medicaments, which are widely prescribed in many indications, the most common being arterial hypertension, but also heart failure, renal insufficiency, nephrotic syndrome, cirrhosis and glaucoma, expose the patient to the risk of a decrease in potassium concentration except for “potassium sparing” diuretics.

A complication of the decrease in potassium concentration subsequent to treatment with diuretics may be sudden death, in particular in patients who present an impairment of the contractile function of the heart or left ventricular dysfunction or after a myocardial infarction.

Diuretics are widely prescribed for their efficacy in the treatment of a diversity of conditions, such as arterial hypertension, congestive heart failure, renal insufficiency, nephrotic syndrome, cirrhosis or glaucoma.

One of the major consequences of a treatment based on diuretics, except for potassium sparing diuretics, is increased potassium excretion which can result in hypokalaemia.

Now, hypokalaemia is known to increase cardiac excitability, resulting, in certain patients, in ventricular arrhythmia and sudden death (Cooper et al., Circulation, 1999, 100, pages 1311-1315).

No combination of an anti-arrhythmic and a diuretic, to date, in therapy, has shown effects with regard to the regulation of the potassium level in the blood and in particular its impact in patients having a history of atrial fibrillation or atrial flutter.

A subject of the present invention is therefore also the use of a combination of dronedarone and of pharmaceutically acceptable salts thereof, with at least one diuretic, in particular a non-potassium-sparing diuretic, for the preparation of a medicament for regulating the potassium level in the blood, in particular for use in the prevention of hypokalaemia.

A subject of the present invention is also the use of a combination of dronedarone and of pharmaceutically acceptable salts thereof, with at least one diuretic, in particular a non-potassium-sparing diuretic, for the preparation of a medicament for use in the prevention of cardiovascular hospitalizations and/or of mortality, in particular cardiovascular mortality and more particularly sudden death in patients having a history of atrial fibrillation or atrial flutter, in particular through the regulation of the potassium level in the blood and more particularly through prevention of hypokalaemia.

A subject of the present invention is therefore also a combination of dronedarone or of a pharmaceutically acceptable salt thereof, with at least one diuretic, with exclusion of furosemide, of hydrochlorothiazide, of metolazone, of amiloride and of spironolactone, and in particular a non-potassium-sparing diuretic, with the exclusion of furosemide, of hydrochlorothiazide and of metolazone.

Said diuretic is administered at therapeutically active doses chosen between 1 mg/day and 2 g/day.

Said combination may be simultaneous, separate or sequential.

Among the pharmaceutically acceptable salts of dronedarone, mention may be made of the hydrochloride.

The term “non-potassium sparing diuretic” is intended to mean a diuretic which increases potassium excretion.

The term “cardiovascular hospitalization” is intended to mean a hospitalization which is caused by at least one of the following pathologies (Hohnloser et al., Journal of cardiovascular electrophysiology, January 2008, vol. 19, No. 1, pages 69-73):
relating to atherosclerosis,
myocardial infarction or unstable angina pectoris,
stable angina pectoris or atypical thoracic pain,
syncope,
transient ischemic event or cerebral stroke (except intracranial haemorrhage),
atrial fibrillation and other supraventricular rhythm disorders,
non-fatal cardiac arrest,
ventricular arrhythmia,
cardiovascular surgery, except heart transplant,
heart transplant,
implantation of a cardiac stimulator (pacemaker), of an implantable defibrillator (“ICD”) or of another cardiac device,
percutaneous coronary, cerebrovascular or peripheral intervention,
variations in arterial pressure (hypotension, hypertension, except syncope),
cardiovascular infection,
major bleeding/haemorrhage (requiring two or more blood cell pellets or any intracranial haemorrhage),
pulmonary embolism or deep vein thrombosis,
worsening of congestive heart failure including acute pulmonary oedema or dyspnoea from cardiac causes.

The term “mortality” covers mortality due to any cause, whether cardiovascular or non-cardiovascular or unknown.

The term “cardiovascular mortality” covers, in the context of the invention, mortality due to any cardiovascular causes (any death except those due to a non-cardiovascular cause), in particular death from an arrhythmic cause, also called arrhythmic death, and more particularly, sudden death from cardiovascular causes, also called sudden death.

The term “sudden death” refers, in general, to death occurring within the hour or less than one hour after the appearance of new symptoms or unexpected death without warning.

It will also be specified that the expression “having a history of atrial fibrillation or atrial flutter”, “having a paroxysmal or persistent atrial fibrillation or atrial flutter” or “having a history of atrial fibrillation or experiencing atrial flutter or atrial fibrillation or of atrial flutter” means a patient who, in the past, has presented one or more episodes of atrial fibrillation or flutter and/or who is suffering from atrial fibrillation or atrial flutter at the time the dronedarone or a pharmaceutically acceptable salt thereof is used.

More particularly, a patient who, in the past, has presented one or more episodes of atrial fibrillation or atrial flutter, may have presented these episodes at least three months or more before random distribution, for example between three and six months.

Among the patients having a history of atrial fibrillation or atrial flutter, mention may also be made of patients also exhibiting at least one of the following risk factors:
age equal to or above 70, more particularly equal to or above 75,
hypertension,
diabetes,
history of cerebral stroke or of systemic embolism,
left atrial diameter greater than or equal to 50 mm measured by echocardiography,
left ventricular ejection fraction less than 40%, measured by two-dimensional echography.

Among the patients having a history of atrial fibrillation or atrial flutter, mention may also be made of patients also exhibiting additional risk factors, i.e. at least one of the following pathologies:
hypertension,
underlying structural heart disease,
tachycardia,
coronary disease,
non-rheumatic heart valve disease,
dilated cardiomyopathy of ischemic origin,
ablation of atrial fibrillation or flutter, for example catheter ablation or endomyocardial ablation,
supraventricular tachycardia other than atrial fibrillation or flutter,
history of heart valve surgery,
non-ischemic dilated cardiomyopathy,
hypertrophic cardiomyopathy,
rheumatic valve disease,
ventricular tachycardia,
cardiopathy,
ablation, for example catheter ablation, for tachycardia other than for atrial fibrillation or flutter,
ventricular fibrillation,

and/or at least one cardiac device chosen from:
a cardiac stimulator,
an implantable defibrillator (“ICD”).

The expression “regulating the potassium level in the blood” is intended to mean preventing the decrease or a possible increase in said level.

The principal classes of non-potassium sparing diuretics are:
thiazide diuretics,
loop diuretics,
proximal diuretics (osmotics, carbonic anhydrase inhibitors).

For their therapeutic use, dronedarone and pharmaceutically acceptable salts thereof are generally introduced into pharmaceutical compositions.

These pharmaceutical compositions contain an effective dose of dronedarone or of a pharmaceutically acceptable salt thereof, and also at least one pharmaceutically acceptable excipient.

Said excipients are chosen according to the pharmaceutical form and the method of administration desired, from the usual excipients which are known to those skilled in the art.

In said pharmaceutical compositions for oral, sublingual, subcutaneous, intramuscular, intravenous, topical, local, intratracheal, intranasal, transdermal or rectal administration, dronedarone, or the salt thereof, can be administered in unit administration form, as a mixture with conventional pharmaceutical excipients, to animals and to humans in the cases mentioned above.

The suitable unit administration forms comprise forms for oral administration, such as tablets, soft or hard gel capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms, and implants. For topical application, dronedarone and pharmaceutically acceptable salts thereof can be used in creams, gels, ointments or lotions.

By way of example, a unit administration form of dronedarone or a pharmaceutically acceptable salt thereof, in tablet form, may correspond to one of the following examples:

| Ingredients | mg | % |
|---|---|---|
| Dronedarone hydrochloride (corresponding to 400 mg | 426 | 65.5 |
| of base) | | |
| Methylhydroxypropylcellulose | 21.1 | 3.25 |
| Lactose monohydrate | 46.55 | 7.2 |
| Maize starch | 45.5 | 7 |
| Polyvinylpyrrolidone | 65 | 10 |
| Poloxamer 407 | 40 | 6.15 |
| Anhydrous colloidal silica | 2.6 | 0.4 |
| Magnesium stearate | 3.25 | 0.5 |
| | 650 | 100 |

| Ingredients | mg | % |
|---|---|---|
| Dronedarone hydrochloride (corresponding to 400 mg | 426 | 65.5 |
| of base) | | |
| Microcrystalline cellulose | 65 | 10 |
| Anhydrous colloidal silica | 2.6 | 0.4 |
| Anhydrous lactose | 42.65 | 6.6 |
| Polyvinylpyrrolidone | 13 | 2 |
| Poloxamer 407 | 40 | 6.15 |
| Macrogol 6000 | 57.5 | 8.85 |
| Magnesium stearate | 3.25 | 0.5 |
| | 650 | 100 |

| Ingredients | mg |
|---|---|
| Dronedarone hydrochloride (corresponding to 400 mg | 426 |
| of base) | |
| Microcrystalline cellulose | 26 |
| Maize starch | 45.5 |
| Polyvinylpyrrolidone | 65 |
| Poloxamer 407 | 40 |
| Anhydrous colloidal silica | 3.25 |
| Magnesium stearate | 3.25 |
| Lactose monohydrate | 41.65 |
| | 650 |

| Ingredients | mg |
|---|---|
| Dronedarone hydrochloride (corresponding to 400 mg | 213 |
| of base) | |
| Microcrystalline cellulose | 13 |
| Maize starch | 22.75 |
| Polyvinylpyrrolidone | 32.5 |
| Poloxamer 407 | 20 |
| Anhydrous colloidal silica | 1.3 |
| Magnesium stearate | 1.625 |
| Lactose monohydrate | 20.825 |
| | 650 |

The dose of dronedarone administered per day, orally, may reach 800 mg, taken in one or more intakes.

More particularly, the dose of dronedarone administered can be taken with food.

More particularly, the dose of dronedarone administered per day, orally, may reach 800 mg, taken in two intakes with a meal.

More particularly, the dose of dronedarone administered per day, orally, can be taken at a frequency of two intakes per day with a meal, for example breakfast and dinner.

More particularly, the two intakes can comprise the same amount.

There may be specific cases where higher or lower dosages are appropriate; such dosages do not depart from the context of the invention. According to the usual practice, the dosage appropriate for each patient is determined by the physician according to the method of administration, the weight, the pathology, the body surface, the cardiac output and the response of said patient.

According to another of its aspects, the present invention also relates to a method for treating the pathologies indicated above, which comprises the administration, to a patient, of an effective dose of dronedarone or a pharmaceutically acceptable salt thereof.

### BRIEF DESCRIPTION OF THE DRAWINGS

The present invention is illustrated by the data hereinafter with reference to the attached drawings in which:

FIG. 1 represents a Kaplan Meier curve with the cumulative rate of hospitalization or of death from any cause over a period of 24 months;

FIG. 2 represents a Kaplan Meier curve with the cumulative rate of hospitalization or of cardiovascular death over a period of 30 months;

FIG. 3 represents a Kaplan Meier curve with the cumulative rate of hospitalization or of sudden death over a period of 30 months;

FIG. 4 represents a Kaplan Meier curve with the cumulative rate of hospitalization over a period of 30 months;

FIG. 5 represents a Kaplan Meier curve with the cumulative rate of death from any cause over a period of 30 months;

FIG. 6 represents a Kaplan Meier curve with the cumulative rate of cardiovascular death over a period of 30 months;

FIG. 7 represents a Kaplan Meier curve with the cumulative rate of sudden death over a period of 30 months;

FIG. 8 represents the mean variations in potassium between the first and the last administration over a period of 30 months.

The efficacy, relative to a placebo, of dronedarone and of pharmaceutically acceptable salts thereof, in the prevention of cardiovascular hospitalizations or of mortality was demonstrated, by means of dronedarone hydrochloride, in a prospective, multinational, multicentre, double-blind clinical study with random distribution in two groups of treatment (group treated with dronedarone hydrochloride and group treated with a placebo) of patients having a history of atrial fibrillation or atrial flutter.

I. Patient Selection

The patients had to have a history of atrial fibrillation or flutter and/or could be in normal sinus rhythm or in atrial fibrillation or flutter at inclusion.

The patient recruitment was carried out by taking into account the following inclusion criteria:

Inclusion Criteria:
1) One of the following risk factors had to be present:
age equal to or above 70, or even above 75, possibly combined with at least one of the risk factors below:
   hypertension (taking antihypertensives of at least two different classes),
   diabetes,
   history of cerebral stroke (transient ischemic event or completed cerebral stroke) or of systemic embolism,
   left atrial diameter greater than or equal to 50 mm measured by echocardiography,
   left ventricular ejection fraction less than 40%, measured by two-dimensional echography;
2) availability of an electrocardiogram carried out during the past 6 months in order to document the presence or the history of atrial fibrillation or flutter;
3) availability of an electrocardiogram carried out during the past 6 months in order to document the presence or absence of normal sinus rhythm.

II. Duration and Treatment

Treatment was initiated using tablets containing either the placebo or an amount of dronedarone hydrochloride corresponding to 400 mg of dronedarone at a rate of one tablet in the morning during or shortly after breakfast and one tablet in the evening during or shortly after dinner.

The anticipated duration of the treatment was variable according to the time at which each patient was included in the study, and could range from a minimum of 12 months for the last patient included up to a maximum corresponding to the entire duration of the study (12 months+duration of inclusion), i.e. approximately 30 months for the first patients included.

III. Results

The results obtained in this trial were analysed by the Kaplan Meier method for the figures, and the relative risk (RR) was estimated using Cox's proportional-effect regression model.

The relative risk (RR) is the ratio of the rates of occurrence of a hospitalization or of a death among the patients on dronedarone, relative to the patients on placebo.

The percentage reduction x of a given event (hospitalization, death, cardiovascular death, etc.) is calculated in the following way:

x=1−relative risk.

III.1. Results Relating to Cardiovascular Hospitalizations and to Mortality (Principal Judgment Criterion)

Among the 4628 patients included in the study, 2301 were part of the group treated with dronedarone hydrochloride.

917 events were recorded in the placebo group, against 734 in the group treated with dronedarone hydrochloride.

The calculated relative risk is 0.758 with a p=2×10⁻⁸, i.e. a reduction in cardiovascular hospitalizations and deaths of 24.2% on dronedarone hydrochloride, the result being highly significant.

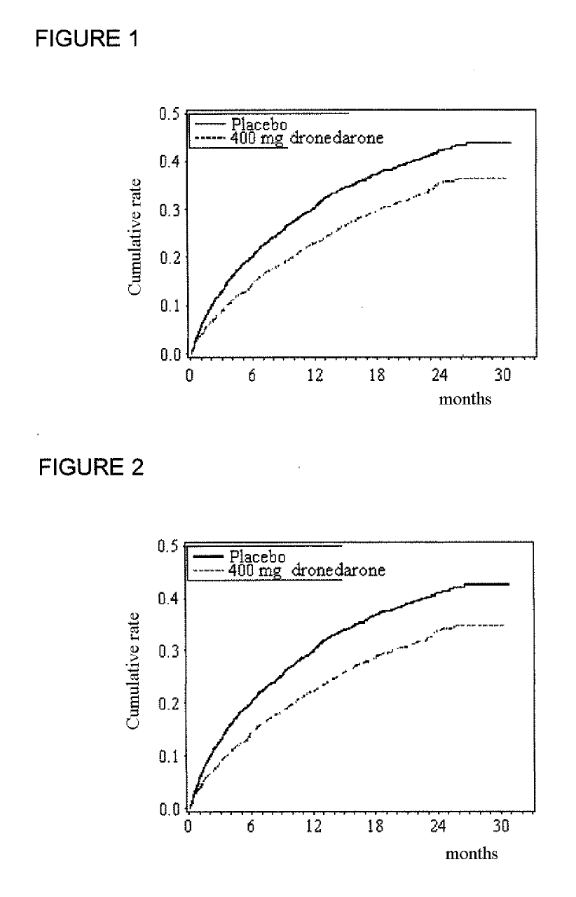

FIG. 1, which reproduces the results obtained, shows a clear separation of the two cumulative curves very soon after the beginning of the treatment, this separation persisting over time throughout the duration of the study.

III.2. Results Relating to Cardiovascular Hospitalizations and to Cardiovascular Mortality

Among the 4628 patients included in the study, 2301 were part of the group treated with dronedarone hydrochloride.

892 events were recorded in the placebo group, against 701 in the group treated with dronedarone hydrochloride.

The calculated relative risk is 0.745 with a p=45×10⁻¹⁰, i.e. a reduction in cardiovascular hospitalizations and cardiovascular deaths of 25.5% on dronedarone hydrochloride, the result being highly significant.

FIG. 2, which reproduces the results obtained, shows a clear separation of the two cumulative curves very soon after the beginning of the treatment, this separation persisting over time throughout the duration of the study.

III.3. Results Relating to Cardiovascular Hospitalizations and to Sudden Death

Among the 4628 patients included in the study, 2301 were part of the group treated with dronedarone hydrochloride.

873 events were recorded in the placebo group, against 684 in the group treated with dronedarone hydrochloride.

The calculated relative risk is 0.743 with a p=48×10⁻¹⁰, i.e. a reduction in cardiovascular hospitalizations and sudden deaths of 25.5% on dronedarone hydrochloride, the result being highly significant.

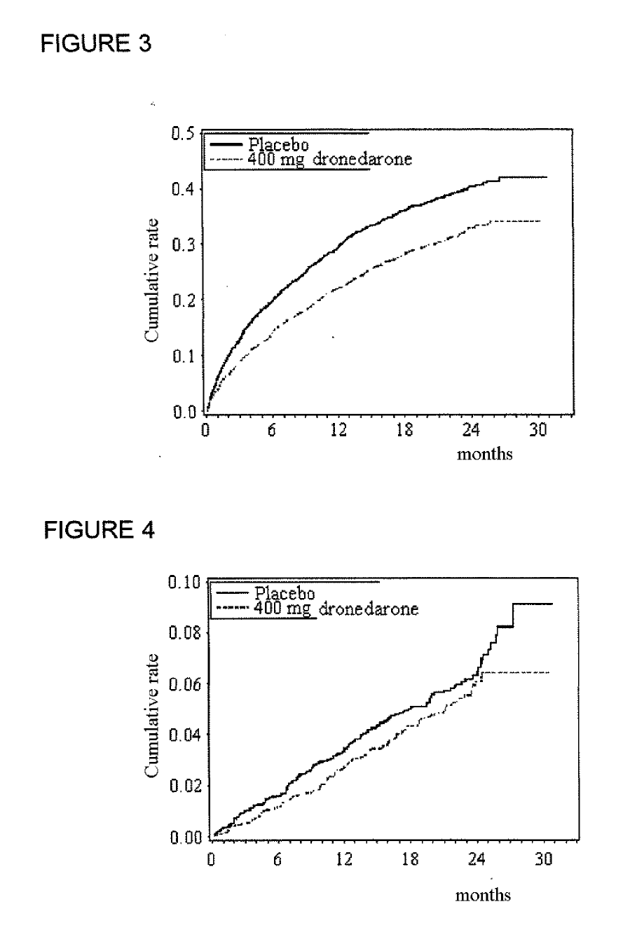

FIG. 3, which reproduces the results obtained, shows a clear separation of the two cumulative curves very soon after the beginning of the treatment, this separation persisting over time throughout the duration of the study.

III.4. Results Relating to Cardiovascular Hospitalizations

Among the 4628 patients included in the study, 2301 were part of the group treated with dronedarone hydrochloride.

859 events were recorded in the placebo group, against 675 in the group treated with dronedarone hydrochloride.

The calculated relative risk is 0.745 with a p=9×10⁻⁹, i.e. a reduction in cardiovascular hospitalizations of 25.5% on dronedarone hydrochloride.

FIG. 4, which reproduces the results obtained, shows a clear separation of the two cumulative curves very soon after the beginning of the treatment, this separation persisting over time throughout the duration of the study.

III.5. Results Relating to Mortality from any Cause

Among the 4628 patients included in the study, 2301 were part of the group treated with dronedarone hydrochloride.

139 deaths were recorded in the placebo group, against 116 in the group treated with dronedarone hydrochloride.

The calculated relative risk is 0.844 with a p=0.1758, i.e. a reduction in cardiovascular hospitalizations of 15.6% on dronedarone hydrochloride.

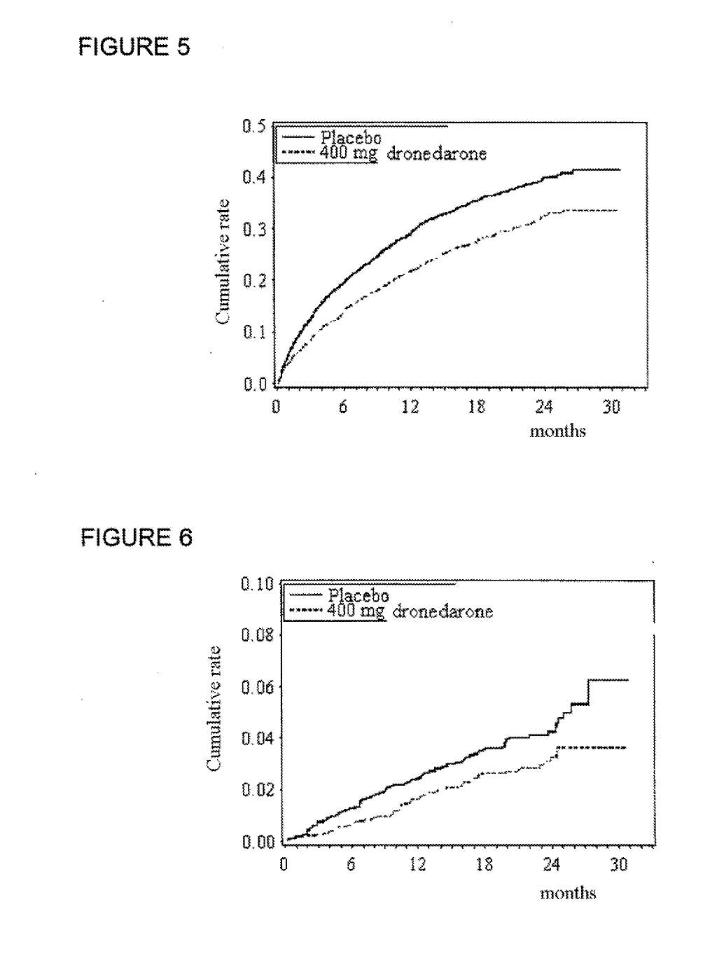

FIG. 5, which reproduces the results obtained, shows a clear separation of the two cumulative curves very soon after the beginning of the treatment, this separation persisting over time throughout the duration of the study.

III.6. Results Relating to Cardiovascular Mortality

Among the 4628 patients included in the study, 2301 were part of the group treated with dronedarone hydrochloride.

94 cardiovascular deaths were recorded in the placebo group, against 65 in the group treated with dronedarone hydrochloride.

The calculated relative risk is 0.698 with a p=0.0252, i.e. a reduction in cardiovascular mortality of 30.2% on dronedarone hydrochloride.

FIG. 6, which reproduces the results obtained, shows a clear separation of the two cumulative curves very soon after the beginning of the treatment, this separation persisting over time throughout the duration of the study.

III.7. Results Relating to Sudden Death

Among the 4628 patients included in the study, 2301 were part of the group treated with dronedarone hydrochloride.

35 sudden deaths were recorded in the placebo group, against 14 in the group treated with dronedarone hydrochloride.

The calculated relative risk is 0.405 with a p=0.0031, i.e. a reduction in sudden death of 59.5% on dronedarone hydrochloride.

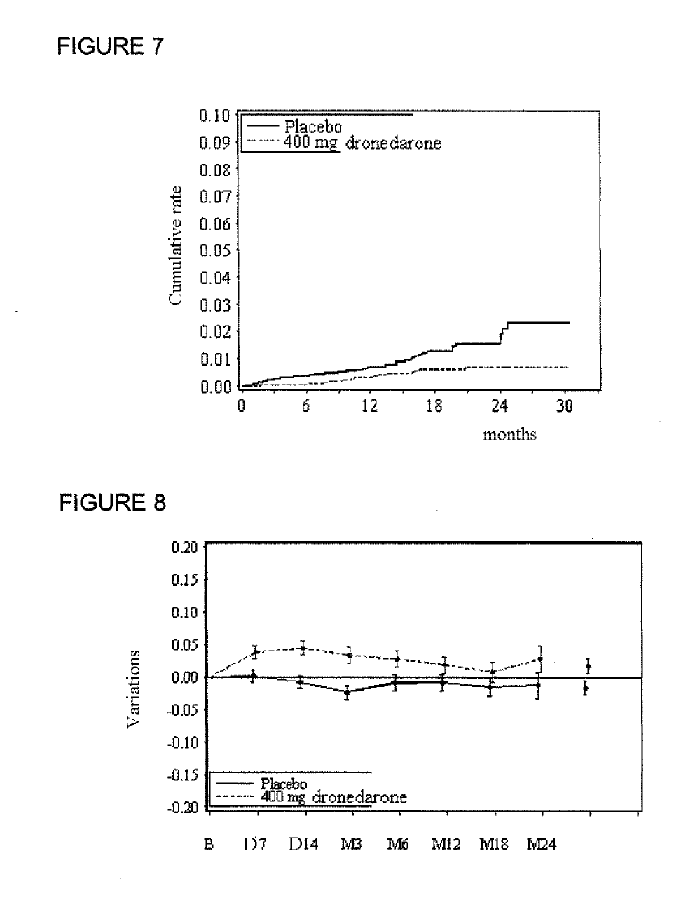

FIG. 7, which reproduces the results obtained, shows a clear separation of the two cumulative curves very soon after the beginning of the treatment, this separation persisting over time throughout the duration of the study.

III.8. Regulation of the Blood Potassium Level

The potassium concentration-modulating effect is clearly documented in the study by virtue of the results of analyses of regular blood samples taken throughout the duration of the study in the context of the monitoring of vital parameters.

The variations in potassium (in mmol/l) between the first and the last administration of the medicament of the study are included in FIG. 8, in which B signifies basal level, D signifies day and M signifies month.

An analysis of covariance of the change in blood potassium level, taking into account the starting value during the study after the 24^{th} month, shows a significant different in favour of dronedarone compared to the placebo (p<0.0001).

Dronedarone therefore makes it possible to regulate the potassium level in the blood.

III.9. Results Relating to the Patients in the Study Receiving, in Addition, a Diuretic-Based Treatment

The clinical results of the study corroborate the hypothesis that modulating potassium decreases the risk of sudden death, in particular in patients exposed to the risk of a decrease in potassium exacerbated by the administration of a diuretic treatment. In fact, the reduction in the risk of sudden death by dronedarone, i.e. the prevention of sudden death compared with the placebo, was approximately 70.4% in the patients on diuretics and approximately 34% in the patients not taking diuretics.

Furthermore, the reduction in the risk was greater in the groups of patients liable to be treated with diuretics, such as hypertensive patients, where the reduction in the risk was approximately 62%, against a reduction of approximately 45.5% observed in the patients who were not hypertensive.

What is claimed is:

1. A method of preventing cardiovascular hospitalizations or mortality in a patient having a history of atrial fibrillation or atrial flutter, comprising administering an effective amount of dronedarone to said patient in combination with one or more diuretic.

2. A method for regulating the potassium level in the blood of a patient, comprising administering an effective amount of dronedarone to said patient in combination with at least one diuretic.

3. A method for treating atrial fibrillation or atrial flutter in patient with a history of atrial fibrillation or atrial flutter, comprising administering an effective amount of dronedarone to said patient in combination with one or more diuretic.

4. The method according to anyone of claims 1 to 3, wherein said diuretic is a non-potassium-sparing diuretic.

5. The method according to claim 4, wherein hypokalaemia is prevented.

6. The method according to anyone of claims 1 to 3, wherein the patient also exhibits at least one risk factors selected from the group consisting of:
hypertension,
diabetes,
history of cerebral stroke or of systemic embolism,
left atrial diameter greater than or equal to 50 mm measured by echocardiography, and
left ventricular ejection fraction less than 40%, measured by two-dimensional echography.

7. The method according to any one of claims 1 to 3, wherein the patient also exhibits at least one pathology selected from the group consisting of:
hypertension,
underlying structural heart disease,
tachycardia,
coronary disease,
non-rheumatic heart valve disease,
dilated cardiomyopathy of ischemic origin,
catheter ablation of atrial fibrillation or flutter,
supraventricular tachycardia other than atrial fibrillation or flutter,
history of valve surgery,
non-ischemic dilated cardiomyopathy,
hypertrophic cardiomyopathy,
rheumatic valve disease,
sustained ventricular tachycardia,
congenital cardiopathy,
catheter ablation for tachycardia other than for atrial fibrillation or flutter,
ventricular fibrillation,
and/or at least one cardiac device chosen from:
a cardiac stimulator, and
an implantable defibrillator (“ICD”).

8. The method according to any one of claims 1 to 3, wherein the dose of dronedarone administered per day, orally, is up to 800 mg, taken in one or more intakes.

9. A combination of dronedarone, or a pharmaceutically acceptable salt thereof, and at least one diuretic, with the exclusion of furosemide, of hydrochlorothiazide, of metolazone, of amiloride, of spironolactone and of mannitol.

10. The combination according to claim 9, wherein said at least one diuretic is a non-potassium-sparing diuretic, with the exclusion of furosemide, of hydrochlorothiazide, of metolazone and of mannitol.
